SEQUENCE LISTING (8 sequences)

SEQ ID NO: 1 - protein, 400 aa, Rattus norvegicus
MESGLLRPAPVSEVIVLHYNYTGKLRGARYQPGAGLRADAAVCLAVCAFIVLENLAVLLVLGRHPRFHAPMFLLLGSLTLSDLLAGAAYATNILLSGPLTLRLSPALWFAREGGVFVALAASVLSLLAIALERHLTMARRGPAPAASRARTLAMAVAAWGLSLLLGLLPALGWNCLGRLEACSTVLPLYAKAYVLFCVLAFLGILAAICALYARIYCQVRANARRLRAGPGSRRATSSSRSRHTPRSLALLRTLSVVLLAFVACWGPLFLLLLLDVACPARACPVLLQADPFLGLAMANSLLNPIIYTFTNRDLRHALLRLLCCGRGPCNQDSSNSLQRSPSAVGPSGGGLRRCLPPTLDRSSSPSEHSCPQRDGMDTSCSTGSPGAATANRTLVPDATD

SEQ ID NO: 2 - DNA, 1203 bp, Rattus norvegicus
atggagtccgggctactgcggccagcgccggtgagcgaggtcatcgtccttcactacaactacactggcaagctccggggagcgcgctaccagcccggtgccggcctgcgtgcggacgccgcagtgtgcttggctgtgtgcgctttcatcgtgctggagaacctggctgtgctcttggtgctgggccgccatcctcgcttccatgcacccatgttcctgctcctgggtagtcttaccttgtcggacctgctcgctggggcggcctacgccaccaacatcctgctgtccgggccgctcacattgcgcctgtcgcctgcgctctggtttgcgcgcgaagggggtgtcttcgtggcgctcgcagcgtcggtgctgagcctcctggccattgctctagagcgccaccttaccatggcccgtcgtggacccgcgcctgccgccagtcgcgctcgcacgctggcgatggcggtggccgcctggggcttgtcgctgctgctggggctgctgcccgcgctgggctggaactgcttgggacgcctggaagcctgctccaccgtgctgccgctctacgccaaggcctatgtgctcttctgcgtgctggccttcctgggcatcctggctgccatctgtgcgctctatgcaaggatttactgtcaggtgcgggccaacgcgcgtcgcctgcgggccggtcctgggtcccgtagggccacgtcctcctcgcgatcccggcacacgccacggtcgttggccctgctccgcacgcttagcgtggtgctcctggccttcgtggcctgctggggacctctatttctcttgctattactggatgtcgcgtgcccagcccgcgcgtgtcctgtgcttctgcaagccgatcccttcctgggtctagccatggctaactcgctgctgaatcctatcatctacaccttcaccaaccgagacctgcgccacgcgctcctgaggctgctctgctgtggccgcggaccctgcaaccaagactcctccaacagtttgcagcgatccccaagtgctgttgggccttccggtggaggcctgcgacgctgcctgccaccaaccctggatcgcagctctagcccatcagaacactcgtgtccccagcgggacgggatggacaccagctgctccactggcagtcccggagcagcaaccgccaaccggaccctggtgcctgatgctacagactga

SEQ ID NO: 3 - DNA, 22 bp, Rattus norvegicus
atttaggtgacactatagaata

SEQ ID NO: 4 - DNA, 19 bp, Rattus norvegicus
gctgcggaaggagtagatg

SEQ ID NO: 5 - DNA, 20 bp, Rattus norvegicus
cctctagatgcatgctcgag

SEQ ID NO: 6 - DNA, 19 bp, Rattus norvegicus
caggagcaggccaaacagg

SEQ ID NO: 7 - protein, 382 aa, Mus musculus
MVSTSIPEVKALRSSVSDYGNYDIIVRHYNYTGKLNIGAEKDHGIKLTSVVFILICCFIILENIFVLLTIWKTKKFHRPMYYFIGNLALSDLLAGVAYTANLLLSGATTYKLTPAQWFLREGSMFVALSASVFSLLAIAIERYITMLKMKLHNGSNSSRSFLLISACWVISLILGGLPSMGWNCISSLSSCSTVLPLYHKHYILFCTTVFTLLLLSIAILYCRIYSLVRTRSRRLTFRKNISKGSRSSEKSLALLKTVIIVLSVFIACWAPLFILLLLDVGCKAKTCDILYKAEYFLVLAVLNSGTNPIIYTLTNKEMRRAFIRIVSCCKCPNGDSAGKFKRPIIPGMEFSRSKSDNSSHPQKDDGDNPETIMSSGNVNSSS

SEQ ID NO: 8 - protein, 383 aa, Rattus norvegicus
MVSSTSIPVVKALRSQVSDYGNYDIIVRHYNYTGKLNIGVEKDHGIKLTSVVFILICCLIILENIFVLLTIWKTKKFHRPMYYFIGNLALSDLLAGVAYTANLLLSGATTYKLTPAQWFLREGSMFVALSASVFSLLAIAIERYITMLKMKLHNGSNSSRSFLLISACWVISLILGGLPIMGWNCISSLSSCSTVLPLYHKHYILFCTTVFTLLLLSIVILYCRIYSLVRTRSRRLTFRKNISKASRSSEKSLALLKTVIIVLSVFIACWAPLFILLLLDVGCKAKTCDILYKAEYFLVLAVLNSGTNPIIYTLTNKEMRRAFIRIISCCKCPNGDSAGKFKRPIIPGMEFSRSKSDNSSHPQKDDGDNPETIMSSGNVNSSS

(19) United States
(12) Patent Application Publication
(10) Pub. No.: US 2005/0191295 A1
(43) Pub. Date: Sep. 1, 2005

(54) Receptor

(76) Inventors: Sultan Ahmad, Ile Perrot (CA); Cyrla Hoffert, Montreal (CA); Dajan O'Donnell, Montreal (CA); Manon Pelletier, Montreal (CA); Philippe Walker, Montreal (CA)

Correspondence Address: FITCH, EVEN, TABIN & FLANNERY, P. O. BOX 65973, WASHINGTON, DC, 20035, US

(21) Appl. No.: 11/100,593

(22) Filed: Apr. 7, 2005

Related U.S. Application Data
(63) Continuation of application No. 09/647,481, filed on Sep. 29, 2000, now abandoned.

(30) Foreign Application Priority Data
Jul. 21, 1999 (SE) 9902763-3

(51) Int. Cl. C12Q 1/68; G01N 33/53; G01N 33/567; C07H 21/04; C07K 14/705; C07K 16/28

(52) U.S. Cl. 424/143.1; 435/6; 435/69.1; 435/320.1; 435/325; 530/350; 530/388.22; 536/23.5

(57) ABSTRACT

The present invention is directed to a novel G protein-coupled receptor expressed, inter alia, in the central nervous system of rats. The invention encompasses the receptor protein as well as nucleic acids encoding the protein. In addition, the invention is directed to methods and compositions which utilize the receptor.

### FIELD OF THE INVENTION

The present invention relates to nucleic acids encoding a novel G protein-coupled receptor and to the receptor itself.

### BACKGROUND OF THE INVENTION

G protein-coupled receptors (GPCRs) constitute a family of proteins sharing a common structural organization characterized by an extracellular N-terminal end, seven hydrophobic alpha helices putatively constituting transmembrane domains and an intracellular C-terminal domain. GPCRs bind a wide variety of ligands that trigger intracellular signals through the activation of transducing G proteins (Caron et al., Rec. Prog. Horm. Res. 48:277-290 (1993); Freedman et al., Rec. Prog. Horm. Res. 51:319-353 (1996)).

Approximately 50-60% of all clinically relevant drugs act by modulating the functions of various GPCRs (Cudermann et al., J. Mol. Med. 73:51-63 (1995)). Of particular interest are receptors located in the central nervous system. G protein-coupled receptors in this region are known to be involved in the transmission, modulation and sensation of pain. Thus, G protein-coupled receptors derived from the brain and spinal column may be used in assays for the identification of new anesthetic and analgesic agents.

### SUMMARY OF THE INVENTION

The present invention is based upon the discovery of a novel G protein-coupled receptor that is structurally distinct from previously reported receptors. It is referred to herein as the “B1C3 receptor.”

In its first aspect, the invention is directed to a protein, except as existing in nature, comprising an amino acid sequence consisting functionally of SEQ ID NO:1. The term “consisting functionally of” refers to proteins in which the sequence of SEQ ID NO:1 has undergone additions, deletions or substitutions which do not substantially alter the functional characteristics of the receptor. The term is intended to encompass proteins having exactly the same amino acid sequence as that of SEQ ID NO:1, as well as proteins with sequence differences that are not substantial as evidenced by their retaining the basic, qualitative ligand binding and physiological properties of the B1C3 receptor. The term “except as existing in nature” refers to a compound that is either expressed by recombinant means or that is in a purified (preferably substantially purified) state.

In a preferred embodiment the protein has an amino acid sequence consisting essentially of the sequence of SEQ ID NO:1. The invention includes antibodies that bind preferentially to such a protein (i.e., antibodies having at least a 100-fold greater affinity for B1C3 than any other protein); and antibodies made by a process involving the injection of a pharmaceutically acceptable preparation of B1C3 into an animal capable of antibody production. Preferably, monoclonal antibody to B1C3 is produced by administering B1C3 to a mouse and then fusing the mouse's spleen cells with myeloma cells.

The invention is also directed to a polynucleotide, except as existing in nature, encoding a protein with an amino acid sequence consisting functionally of SEQ ID NO:1. This aspect of the invention encompasses polynucleotides encoding proteins consisting essentially of the amino acid sequence of SEQ ID NO:1, expression vectors comprising such polynucleotides, and host cells transformed with such vectors. Also included is recombinant B1C3 receptor produced by host cells made in this manner. Preferably, the polynucleotide encoding the B1C3 receptor has a sequence consisting essentially of the nucleotide sequence of SEQ ID NO:2, and the vectors and host cells used for expression of the receptor also use this particular polynucleotide.

In another aspect, the present invention is directed to a method for assaying a test compound for its ability to bind to the B1C3 receptor. The method is performed by incubating a source of B1C3 with a ligand known to bind to the receptor and with the test compound. The source of receptor should, preferably, express a large amount of B1C3 relative to other G protein-coupled receptors. Upon completion of incubation, the ability of the test compound to bind to B1C3 is determined by the extent to which ligand binding has been displaced. Preferably, the receptor present should have the sequence shown in SEQ ID NO:1. Although not essential, the binding assay can be accompanied by an assay to determine whether a second messenger pathway, e.g., the adenyl cyclase pathway, has become activated. This should help to determine whether a particular compound binding to B1C3 is acting as an agonist or antagonist.

A second method for determining if a test compound is an B1C3 agonist, a method that does not require any ligand, is to use a cell signaling assay, e.g., an assay measuring either intracellular adenyl cyclase activity or intracellular calcium concentration. The test compound should generally be incubated with cells expressing high amounts of B1C3 relative to other G protein-coupled receptors, typically a cell transfected with an expression vector encoding the B1C3 of SEQ ID NO:1. Test compounds that are agonists are identified by their causing a statistically significant change in the results obtained from the cell signaling assay when compared to control cells not exposed to test compound. The control cells may be either cells that have not been transfected or cells that have been mock transfected with a vector that does not produce active receptor. B1C3-expressing cells exposed to test compounds that are agonists would typically be expected to show a significant increase in adenyl cyclase activity or in intracellular calcium concentration relative to control cells.

It is a well known that, upon stimulation with an agonist, GPCRs internalize. Thus, another method that can be used to identify an endogenous ligand of B1C3, or to screen for compounds that act as agonists of the receptor, is to determine whether a cell expressing the receptor internalizes it in response to contact with one or more test compounds. One method of accomplishing this is to label the B1C3 receptor with a fluorescent probe (e.g., by expressing it as a fusion protein linked to the green fluorescent protein) and use microscopy to determine whether internalization occurs (see Example 3). In doing this, one may start with a crude extract of compounds and, if internalization occurs, purify the factors responsible using standard methods of biochemistry.

The invention also encompasses a method for determining if a test compound is an antagonist or inverse agonist of B1C3 which relies upon the known constitutive activation of G protein-coupled receptors that occurs when such receptors are expressed in large amounts. This method requires that DNA encoding the receptor be incorporated into an expression vector so that it is operably linked to a promoter and that the vector then be used to transfect an appropriate host. In order to produce sufficient receptor to result in constitutive receptor activation (i.e., activation in the absence of natural ligand), expression systems capable of copious protein production are preferred, e.g., the B1C3 DNA may be operably linked to a CMV promoter and expressed in COS or HEK293 cells. After transfection, cells with activated receptors are selected based upon their showing increased activity in a cell signaling assay relative to comparable cells that have either not been transfected or that have been transfected with a vector that is incapable of expressing functional B1C3. Typically, cells will be selected either because they show a statistically significant change in intracellular adenyl cyclase activity, in intracellular calcium concentration, or in repoter gene activity such as SEAP (secreted alkaline phosphatase). The selected cells are contacted with the test compound and the cell signaling assay is repeated to determine if this results in a decrease in activity relative to selected cells that have not been contacted with the test compound. For example, a statistically significant decrease in either adenyl cyclase activity, calcium concentration or SEAP actvity relative to control cells would indicate that the test compound is an antagonist of B1C3. Preferably the B1C3 used in assays has the sequence of SEQ ID NO:1.

Assays for compounds interacting with B1C3 may be performed by incubating a source containing the receptor (e.g., a stably transformed cell) with a ligand specific for B1C3 both in the presence and absence of test compound and measuring the modulation of intracellular calcium concentration. A significant increase or decrease in ligand-stimulated calcium signaling in response to test compound is indicative of an interaction occurring at the B1C3 receptor. The preferred receptor is that having the amino acid sequence of SEQ ID NO:1.

In another aspect, the present invention is directed to a method for assaying a test compound for its ability to alter the expression of B1C3. This method is performed by growing cells expressing B1C3 in the presence of the test compound. Cells are then collected and the expression of B1C3 is compared with expression in control cells grown under essentially identical conditions but in the absence of test compound. The preferred receptor is one having the amino acid sequence of SEQ ID NO:1. A preferred test compound is an oligonucleotide at least 15 nucleotides in length comprising a sequence complementary to the sequence of the B1C3 mRNA used in the assay.

### BRIEF DESCRIPTION OF THE DRAWINGS

FIG. 1. FIG. 1 contains the complete nucleotide sequence of a clone constructed by the methods described in the Examples section. The clone was deposited with the International Depository Authority Deutsche Sammlung Von Mikroorganismen Und Zellkulturen GmbH at the address Mascheroder Weg 1 B, D-3300 Braunschweig, Germany. The deposit was made on May 14, 1999 and was given the accession number DSM 12808.

FIG. 2. FIG. 2 shows the deduced amino acid sequence of rat B1C3. The polynucleotide of FIG. 1 codes for a protein 400 amino acids in length.

FIG. 3. FIG. 3 shows a sequence comparison between B1C3 and sequences for mouse and rat EDG-1 receptors. B1C3 is about 34% homologous to the mouse EDG-1 receptor and about 33% homologous to the rat EDG-1 receptor.

### DEFINITIONS

The description that follows uses a number of terms that refer to recombinant DNA technology. In order to provide a clear and consistent understanding of the specification and claims, including the scope to be given such terms, the following definitions are provided.

Cloning vector: A plasmid or phage DNA or other DNA sequence which is able to replicate autonomously in a host cell and which is characterized by one or a small number of restriction endonuclease recognition sites. A foreign DNA fragment may be spliced into the vector at these sites in order to bring about the replication and cloning of the fragment. The vector may contain a marker suitable for use in the identification of transformed cells. For example, a marker may provide tetracycline resistance or ampicillin resistance.

Expression vector: A vector similar to a cloning vector but which is capable of inducing the expression of the DNA that has been cloned into it, after transformation into a host. The cloned DNA is usually placed under the control of (i.e., operably linked to) certain regulatory sequences such as promoters or enhancers. Promoters may be constitutive, inducible or repressible.

Substantially pure: As used herein, “substantially pure” means that the desired product is essentially free from contaminating cellular components. A “substantially pure” protein or nucleic acid will typically comprise at least 85% of a sample, with greater percentages being preferred. Contaminants may include proteins, carbohydrates or lipids. One method for determining the purity of a protein or nucleic acid is by electrophoresing a preparation in a matrix such as polyacrylamide or agarose. Purity is evidenced by the appearance of a single band after staining. Other methods for assessing purity include chromatography and analytical centrifugation.

Recombinant protein: A recombinant protein or recombinant receptor is a non-endogenous protein produced by the introduction of an expression vector into host cells.

Host: Any prokaryotic or eukaryotic cell that is the recipient of a replicable expression vector or cloning vector is the “host” for that vector. The term encompasses prokaryotic or eukaryotic cells that have been engineered to incorporate a desired gene on its chromosome or in its genome. Examples of cells that can serve as hosts are well known in the art, as are techniques for cellular transformation (see, e.g., Sambrook et al., Molecular Cloning: A Laboratory Manual, 2nd ed. Cold Spring Harbor (1989)).

Promoter: A DNA sequence typically found in the 5′ region of a gene, located proximal to the start codon. Transcription is initiated at the promoter. If the promoter is of the inducible type, then the rate of transcription increases in response to an inducing agent.

Complementary Nucleotide Sequence: A complementary nucleotide sequence, as used herein, refers to the sequence that would arise by normal base pairing. For example, the nucleotide sequence 5′-AGAC-3′ would have the complementary sequence 5′-GTCT-3′.

Expression: Expression is the process by which a polypeptide is produced from DNA. The process involves the transcription of the gene into mRNA and the translation of this mRNA into a polypeptide.

### DETAILED DESCRIPTION OF THE INVENTION

The present invention is directed to an R1C3 receptor protein, genetic sequences coding for the protein, methods for assaying compounds to determine if they interact with B1C3 and a method for assaying compounds for their ability to alter receptor expression.

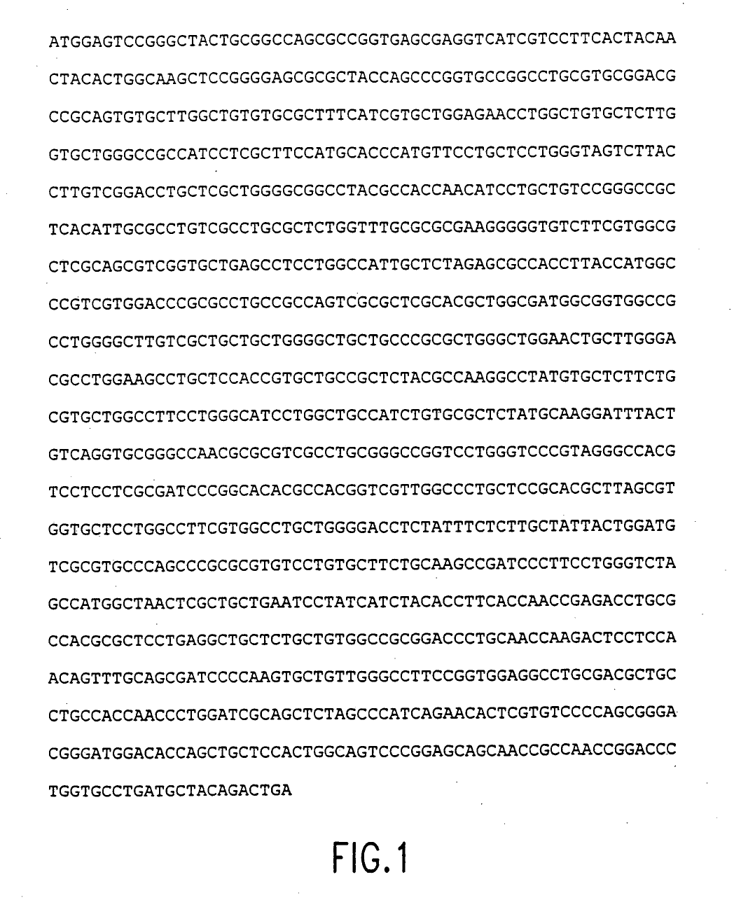

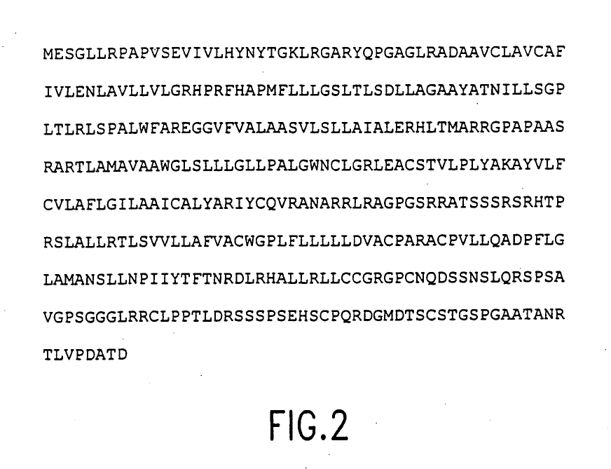

The nucleic acid encoding the receptor and the receptor itself are defined by the structures shown in FIG. 1 (SEQ ID NO:2) and FIG. 2 (SEQ ID NO:1) respectively. However, the invention encompasses not only sequences identical to those shown in the figures and sequence listing, but also sequences that are essentially the same and sequences that are otherwise substantially the same and which result in a receptor retaining the basic binding characteristics of B1C3. For example, it is well known that techniques such as site-directed mutagenesis may be used to introduce variations into a protein's structure. Variations in the B1C3 receptor introduced by this or some similar method are encompassed by the invention provided that the resulting receptor retains the basic qualitative binding and physiological characteristics of unaltered B1C3. Thus, the invention relates to proteins comprising amino acid sequences consisting functionally of SEQ ID NO:1.

I. Nucleic Acid Sequences Coding for B1C3

DNA sequences coding for rat B1C3 are expressed throughout the adult rat central nervous system. Tissue from these areas may be used as a source for the isolation of nucleic acids coding for the receptor. In addition, cells and cell lines that express B1C3 may be used. These may either be cultured cells that have not undergone transformation or cell lines specifically engineered to express recombinant B1C3. Most preferred are the cells deposited as DSMZ No. 12808. DNA encoding B1C3 may be obtained as the result of standard restriction digestions. Alternatively, poly A⁺mRNA may be isolated from tissue or cells, reverse transcribed and cloned. The cDNA library thus formed may then be screened using probes derived from SEQ ID NO:2. Probes should typically be at least 14 bases in length and should, preferably, not be obtained from regions of the DNA corresponding to highly conserved transmembrane domains of B1C3.

B1C3 can also be obtained from recombinant cells containing the full-length B1C3 sequence or from cDNA libraries by performing PCR amplifications using primers located at either end of the B1C3 gene. These primers can be selected from the sequences shown in SEQ ID NO:2.

II. Antibodies to B1C3

The present invention is also directed to antibodies that bind specifically to B1C3 and to a process for producing such antibodies. Antibodies that “bind specifically” are defined as those that have at least a one hundred fold greater affinity for B1C3 than for other proteins. The process for producing such antibodies may involve either injecting the B1C3 protein itself into an appropriate animal or, alternatively, injecting short peptides made to correspond to different regions of the receptor. The peptides should be at least five amino acids in length and should be selected from regions believed to be unique to B1C3. Thus, highly conserved transmembrane regions should generally be avoided in selecting peptides for the generation of antibodies. Methods for making and detecting antibodies are well known to those of skill in the art as evidenced by standard reference works such as: Harlow et al., Antibodies. A Laboratory Manual, Cold Spring Harbor Laboratory, N.Y (1988)); Klein, Immunology: The Science of Self-Nonself Discrimination (1982); Kennett et al., Monoclonal Antibodies and Hybridomas: A New Dimension in Biological Analyses (1980); and Campbell, “Monoclonal Antibody Technology,” in Laboratory Techniques in Biochemistry and Molecular Biology, (1984)).

“Antibody,” as used herein, is meant to include intact molecules as well as fragments which retain their ability to bind to antigen (e.g., Fab and F(ab′)₂ fragments). These fragments are typically produced by proteolytically cleaving intact antibodies using enzymes such as papain (to produce Fab fragments) or pepsin (to produce F(ab′)₂ fragments). The term “antibody” also refers to both monoclonal antibodies and polyclonal antibodies. Polyclonal antibodies are derived from the sera of animals immunized with the antigen. Monoclonal antibodies can be prepared using hybridoma technology (Kohler et al., Nature 256:495 (1975); Hammerling et al., in: Monoclonal Antibodies and T-Cell Hybridomas, Elsevier, M.Y, pp. 563-681 (1981)). In general, this technology involves immunizing an animal, usually a mouse, with either intact B1C3 or a fragment derived from B1C3. The splenocytes of the immunized animals are extracted and fused with suitable myeloma cells, e.g., SP₂O cells. After fusion, the resulting hybridoma cells are selectively maintained in HAT medium and then cloned by limiting dilution (Wands et al., Gastroenterology 80:225-232 (1981)). The cells obtained through such selection are then assayed to identify clones which secrete antibodies capable of binding to B1C3.

The antibodies, or fragments of antibodies, of the present invention may be used to detect the presence of B1C3 using any of a variety of immunoassays. For example, the antibodies may be used in radioimmunoassays or in immunometric assays, also known as “two-site” or “sandwich” assays (see Chard, T., “An Introduction to Radioimmune Assay and Related Techniques,” in Laboratory Techniques in Biochemistry and Molecular Biology, North Holland Publishing Co., N.Y. (1978)). In a typical immunometric assay, a quantity of unlabeled antibody is bound to a solid support that is insoluble in the fluid being tested, e.g., blood, lymph, cellular extracts, etc. After the initial binding of antigen to immobilized antibody, a quantity of detectably labeled second antibody (which may or may not be the same as the first) is added to permit detection and/or quantitation of bound antigen (see, e.g., Radioimmune Assay Method, Kirkham et al., ed., pp. 199-206, E & S. Livingstone, Edinburgh (1970)). Many variations of these types of assays are known in the art and may be employed for the detection of B1C3.

Antibodies to B1C3 may also be used in the purification of either intact receptor or fragments of the receptor (see generally, Dean et al., Affinity Chromatography, A Practical Approach, IRL Press (1986)). Typically, antibody is immobilized on a chromatographic matrix such as Sepharose 4B. The matrix is then packed into a column and the preparation containing B1C3 is passed through under conditions that promote binding, e.g., under conditions of low salt. The column is then washed and bound B1C3 is eluted using a buffer that promotes dissociation from antibody, e.g., buffer having an altered pH or salt concentration. The eluted B1C3 may be transferred into a buffer of choice, e.g., by dialysis, and either stored or used directly.

III. Radioligand Assay for Receptor Binding

One of the main uses for B1C3 nucleic acids and recombinant proteins is in assays designed to identify agents capable of binding to the receptor. Such agents may either be agonists, mimicking the normal effects of receptor binding, or antagonists, inhibiting the normal effects of receptor binding. Of particular interest is the identification of agents which bind to the B1C3 receptor and modulate intracellular signaling, such as adenyl cyclase activity or intracellular calcium. These agents have potential therapeutic application as either analgesics or anesthetics.

In radioligand binding assays, a source of B1C3 is incubated together with a ligand known to bind to the receptor and with the compound being tested for binding activity. The preferred source of B1C3 is cells, preferably mammalian cells, transformed to recombinantly express the receptor. The cells selected should not express a substantial amount of any other G protein-coupled receptor that might bind to ligand and distort results. This can easily be determined by performing binding assays on cells derived from the same tissue or cell line as those recombinantly expressing B1C3 but which have not undergone transformation.

The assay may be performed either with intact cells or with membranes prepared from the cells (see, e.g., Wang et al., Proc. Natl. Acad. Sci. U.S.A. 90:10230-10234 (1993)). The membranes, or cells, are incubated with a ligand specific for the B1C3 receptor and with a preparation of the compound being tested. After binding is complete, receptor is separated from the solution containing ligand and test compound, e.g., by filtration, and the amount of binding that has occurred is determined. Preferably, the ligand used is detectably labeled with a radioisotope such as ¹²⁵I. However, if desired, fluorescent or chemiluminescent labels can be used instead. Among the most commonly used fluorescent labeling compounds are fluorescein isothiocynate, rhodamine, phycoerythrin, phycocyanin, allophycocyanin, o-phthaldehyde and fluorescamine. Useful chemiluminescent compounds include luminol, isoluminol, theromatic acridinium ester, imidazole, acridinium salt, and oxalate ester. Any of these agents can be used to produce a ligand suitable for use in the assay.

Nonspecific binding may be determined by carrying out the binding reaction in the presence of a large excess of unlabeled ligand. For example, labeled ligand may be incubated with receptor and test compound in the presence of a thousandfold excess of unlabeled ligand. Nonspecific binding should be subtracted from total binding, i.e. binding in the absence of unlabeled ligand, to arrive at the specific binding for each sample tested. Other steps such as washing, stirring, shaking, filtering and the like may be included in the assays as necessary. Typically, wash steps are included after the separation of membrane-bound ligand from ligand remaining in solution and prior to quantitation of the amount of ligand bound, e.g., by counting radioactive isotope. The specific binding obtained in the presence of test compound is compared with that obtained in the presence of labeled ligand alone to determine the extent to which the test compound has displaced receptor binding.

In performing binding assays, care must be taken to avoid artifacts which may make it appear that a test compound is interacting with the B1C3 receptor when, in fact, binding is being inhibited by some other mechanism. For example, the compound being tested should be in a buffer which does not itself substantially inhibit the binding of ligand to B1C3 and should, preferably, be tested at several different concentrations. Preparations of test compound should also be examined for proteolytic activity and it is desirable that antiproteases be included in assays. Finally, it is highly desirable that compounds identified as displacing the binding of ligand to B1C3 receptor be reexamined in a concentration range sufficient to perform a Scatchard analysis of the results. This type of analysis is well known in the art and can be used for determining the affinity of a test compounds for receptor (see, e.g., Ausubel et al., Current Protocols in Molecular Biology, 11.2.1-11.2.19 (1993); Laboratory Techniques in Biochemistry and Molecular Biology, Work et al., ed., N.Y. (1978) etc.). Computer programs may be used to help in the analysis of results (see, e.g., Munson, P., Methods Enzymol. 92:543-577 (1983); McPherson, G. A., Kinetic, EBDA Ligand, Lowry-A Collection of Radioligand Binding Analysis Programs, Elsevier-Biosoft, U.K. (1985)).

The activation of receptor by the binding of ligand may be monitored using a number of different assays. For example, adenyl cyclase assays may be performed by growing cells in wells of a microtiter plate and then incubating the wells in the presence or absence of test compound. cAMP may then be extracted in ethanol, lyophilized and resuspended in assay buffer. Assay of cAMP thus recovered may be carried out using any method for determining cAMP concentration, e.g. the Biotrack cAMP Enzyme-immunoassay SystemJ (Amersham) or the Cyclic AMP [³H] Assay System (Amersham). Typically, adenyl cyclase assays will be performed separately from binding assays, but it may also be possible to perform binding and adenyl cyclase assays on a single preparation of cells. Other “cell signaling assays” that can be used to monitor receptor activity are described below.

IV. Identification of B1C3 Agonists and Antagonists Using Cell Signaling Assays

B1C3 receptors may also be used to screen for drug candidates using cell signaling assays. To identify B1C3 agonists, the DNA encoding a receptor is incorporated into an expression vector and then transfected into an appropriate host. The transformed cells are then contacted with a series of test compounds and the effect of each is monitored. Among the assays that can be used are assays measuring cAMP production (see discussion above), assays measuring the activation of reporter gene activity, assays measuring the modulation of the binding of ligand, e.g., GTP-gamma-S, or assays measuring changes in intracellular calcium concentration.

Cell signaling assays may also be used to identify B1C3 antagonists. G protein-coupled receptors can be put into their active state even in the absence of their cognate ligand by expressing them at very high concentration in a heterologous system. For example, receptor may be overexpressed using the baculovirus infection of insect Sf9 cells or the B1C3 gene may be operably linked to a CMV promoter and expressed in COS or HEK293 cells. In this activated constitutive state, antagonists of the receptor can be identified in the absence of ligand by measuring the ability of a test compound to inhibit constitutive cell signaling activity. Appropriate assays for this are, again, cAMP assays, reporter gene activation assays or assays measuring the binding of GTP-gamma-S.

One preferred cell signaling assay is based upon cells stably transfected with B1C3 showing a change in intracellular calcium levels in response to incubation in the presence of ligand. Thus, a procedure can be used to identify B1C3 agonists or antagonists that is similar to the radioreceptor assays discussed above except that calcium concentration is measured instead of bound radioactivity. The concentration of calcium in the presence of test compound and ligand is compared with that in the presence of ligand alone to determine whether the test compound is interacting at the B1C3 receptor. A statistically significant increase in intracellular calcium in response to test compound indicates that the test compound is acting as an agonist whereas a statistically significant decrease in intracellular calcium indicates that it is acting as an antagonist.

Assays may also be performed that measure the activation of a reporter gene. For example, cells expressing recombinant B1C3 may be transfected with a reporter gene (e.g., a chloramphenicol acetyltransferase or luciferase gene) operably linked to an adenyl cyclase or diacylglycerol response element. The cells are then incubated with test compounds and the expression of the reporter gene is compared to expression in control cells that do not express recombinant B1C3 but that are essentially identical in other respects. A statistically significant change in reporter gene expression in the B1C3-expressing cells is indicative of a test compound that interacts with the B1C3 receptor.

V. Assay for Ability to Modulate B1C3 Expression

One way to either increase or decrease the biological effects of B1C3 is to alter the extent to which the receptor is expressed in cells. Therefore, assays for the identification of compounds that either inhibit or enhance expression are of considerable interest. These assays are carried out by growing cells expressing B1C3 in the presence of a test compound and then comparing receptor expression in these cells with expression in cells grown under essentially identical conditions but in the absence of test compound. As in the binding assays discussed above, it is desirable that the cells used be substantially free of competing G protein-coupled receptors. One way to measure receptor expression is to fuse the B1C3 sequence to a sequence encoding a peptide or protein that can be readily quantitated. For example, the B1C3 sequence may be ligated to a sequence encoding hemagglutinin and used to stably transfect cells. After incubation with test compound, the hemagglutinin/receptor complex can be immuno-precipitated and Western blots performed using anti-hemagglutinin antibody. Alternatively, Scatchard analysis of binding assays may be performed with labeled ligand to determine receptor number. The binding assays may be carried out as discussed above and will preferably utilize cells that have been engineered to recombinantly express B1C3.

A preferred group of test compounds for inclusion in the B1C3 expression assay consists of oligonucleotides complementary to various segments of the B1C3 nucleic acid sequence shown in SEQ ID NO:2. These oligonucleotides should be at least 15 bases in length and should be derived from non-conserved regions of the receptor nucleic acid sequence.

Oligonucleotides which are found to reduce receptor expression may be derivatized or conjugated in order to increase their effectiveness. For example, nucleoside phosphorothioates may be substituted for their natural counterparts (see Cohen, J., Oligodeoxynucleotides, Antisense Inhibitors of Gene Expression, CRC Press (1989)). The oligonucleotides may also be delivered to a patient in vivo for the purpose of inhibiting B1C3 expression. When this is done, it is preferred that the oligonucleotide be administered in a form that enhances its uptake by cells. For example, the oligonucleotide may be delivered by means of a liposome or conjugated to a peptide that is ingested by cells (see, e.g., U.S. Pat. Nos. 4,897,355 and 4,394,448; see also non-U.S. patent documents WO 8903849 and EP 0263740). Other methods for enhancing the efficiency of oligonucleotide delivery are well known in the art and are also compatible with the present invention.

Having now described the invention, the same will be more readily understood through reference to the following Examples which are provided by way of illustration and which are not intended to limit the scope of the invention.

### EXAMPLES

### Example 1

### Cloning of Rat BC13

Generation of Probe for cDNA Library Screening: One method of obtaining a novel sequence encoding a receptor is to perform moderate to low stringency screening of cDNA libraries with a relevant probe. In order to find novel genes related to receptors for lipids (sphingosine phosphate, lysophosphatidic acid, cannabinoids etc) the sequence of an EST (Expressed Sequence Tag), aa451451, was used to generate a cDNA probe for the screening cDNA libraries. The following oligonucleotides were used for PCR:

| | | |
|---|---|---|
| 5′ ATT TAG GTG ACA CTA TAG AAT A 3′ | SP6 primer | (SEQ ID NO:3) |
| 5′ GCT GCG GAA GGA GTA GAT G 3′ | 451-1R primer | (SEQ ID NO:4) |
| 5′ CCT CTA GAT GCA TGC TCG AG 3′ | SP6-1R internal primer | (SEQ ID NO:5) |
| 5′ CAG GAG CAG GCC AAA CAG G 3′ | 451-3R internal primer | (SEQ ID NO:6) |

Two of these oligonucleotides were used in a polymerase chain reaction mixture (total volume 100 μl), which contained 1.5 ìl of a rat brain (minus cerebellum) cDNA library in pcDNA1 (Invitrogen), 1×PCR buffer (14 mM (NH₄)₂SO₄, 1.5 mM MgCl₂, 50 mM Tris-HCl (pH 9.2), Roche Diagnostics), 200 μM dNTPs (Amersham-Pharmacia), 2.6 U Expand High FidelityJ (Roche Diagnostics) and 100 pmoles each of the SP6 and 451-1R primers. Amplification was carried out on a Gene Amp PCR Sys 9700J (Perkin Elmer: Applied Biosystems). The template was denatured at 94° C. for 3 minutes, followed by 35 cycles consisting of denaturation, annealing and extension steps, each for 1 minute at 94° C., 53° C. and 72° C., respectively, and then extended for an additional 5 minutes at 72° C.

The resulting product was diluted 1:100 and amplified with primers SP6-1R and 451-3R. The polymerase chain reaction mixture (50 ìl) contained 1 ìl of diluted solution from the first PCR reaction, 1×PCR buffer (same as above, Roche Diagnostics), 200 ìM dNTPs (Amersham-Pharmacia), 2.6 U Expand High FidelityJ (Roche Diagnostics) and 50 pmoles each of the SP6-1R and 451-3R primers. The amplification was carried out on a Gene Amp PCR Sys 9700J (Perkin Elmer: Applied Biosystems). Template was denatured at 94° C. for 3 minutes, followed by 25 cycles consisting of denaturation, annealing and extension steps at 94° C. (I minute), 53° C. (1 minute) and 72° C. (1 minute 20 seconds) respectively, and then extended for an additional 5 minutes at 72° C.

A PCR product of 450 bp was excised and purified with QIAquickJ gel extraction kit (Qiagen), blunted with Klenow (Amersham-Pharmacia) and subcloned into KS⁻ digested by ER V and dephosphorylated. The plasmids were prepared with the alkaline lysis protocol using a Qiaprep 8J kit (Qiagen) and screened by sequencing using ABI Prism dRhodamineJ cycle sequencing ready reaction kit (PE Applied Biosystems). The sequence of the 450 bp rat fragment was identical to the of EST aa451451. A NotI/HindIII fragment of 400 bp was then digested, excised and purified with QIAquickJ gel extraction kit (Qiagen) to be used as a tool to isolate an EDG related rat clone.

cDNA Library Screening: A Stratagene rat brainstem/spinal cord cDNA library (cat # 936521) was screened using the NotI/HindIII fragment as a probe. 8×10⁵ plaques were plated, transferred to Hybond N+J filters (Amersham-Pharmacia), denatured for 5 minutes (1.5M NaCl and O.5M NaOH) and neutralized for 5 minutes (1.5M NaCl, 0.5M Tris-HCl pH 8). The filters were rinsed in 2×SSC and air-dried for one hour at room temperature. The membranes were hybridized overnight at 42° C. in 50% formamide, 10% dextran sulphate (from a 50% solution), 1% SDS, 5×SSC, 1× Denhardt's, 10 mM Tris pH 8 and 100_g/ml of sonicated salmon sperm. The NotI/HindIII fragment was randomly labeled using T7 Quick PrimeJ (Amersham-Pharmacia) and added to the hybridization solution at 3×10⁶ cpm/ml. The filters were washed with 2×SSC/0.1% SDS at room temperature followed by a high stringency wash with 0.2×SSC/0.1% SDS at 65° C.

Using this screening procedure, 6 independent clones were identified and isolated by repeated plating and screening with the labeled NotI/HindIII fragment. Two independent clones were subjected to sequencing (clones B1A7 and C3A3). The DNA sequence analysis revealed that the clone B1A7 contained the 5′ sequence of a putative novel GPCR but was missing the 3′ end. Although C3A3 contained the whole coding sequence, it was missing one nucleotide, leading to a premature stop codon. In order to obtain the full coding sequence, the 2 clones were digested by XbaI and ligated together. The resulting clone “B1C3” contains the complete coding region of a putative novel GPCR.

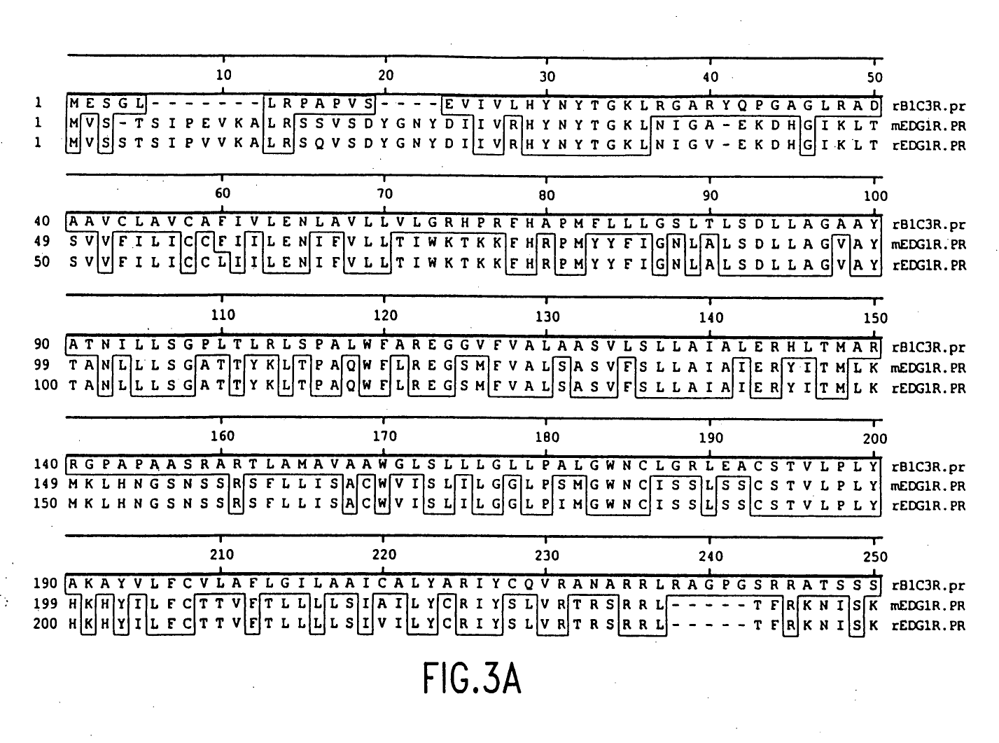

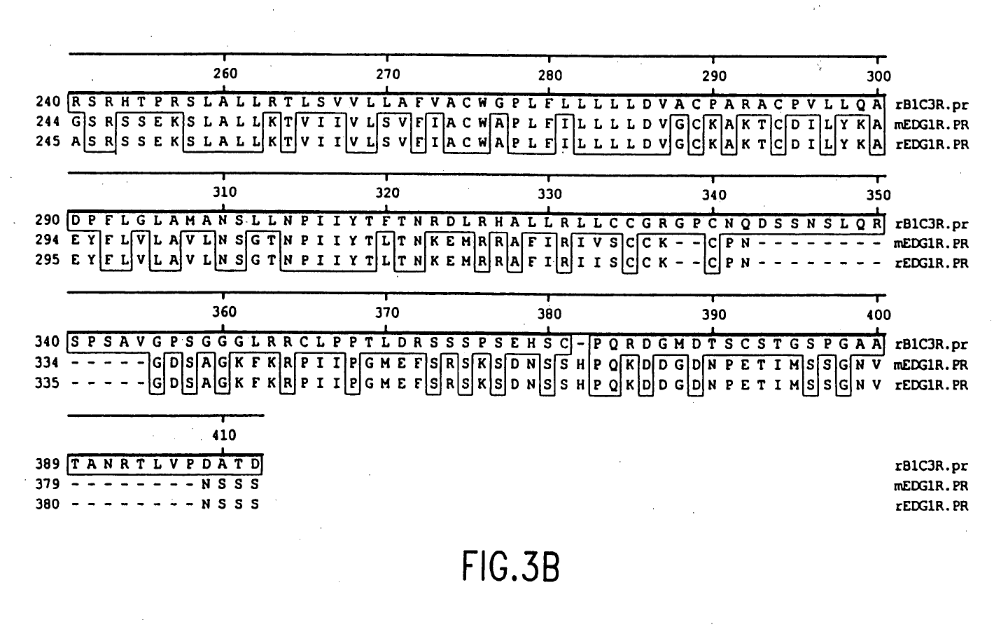

Results: The complete nucleotide sequence of the B1C3 cDNA clone is shown in FIG. 1. The open reading frame is comprised of 1203 nucleotides, encoding a protein of 401 amino acids (FIG. 2) with a predicted molecular mass of about 42.3 kDa. The protein sequence contains hallmark features of GPCRs: the presence of seven hydrophobic helices likely to represent transmembrane domains; an amino terminus; and a carboxy terminus. In addition, several modification sites proposed to be involved in the regulation of receptor function are also present in the predicted amino acid sequence. Thus, the receptor sequence has: a potential cAMP phosphorylation site at position 236; N-linked glycosylation sites; myristilation sites; and Protein Kinase-C phosphorylation sites. The nucleotide sequence and primary predicted amino acid sequence of the B1C3 receptor are not present in their entirety in the Genebank database. The sequences most closely resemble those of the mouse and rat EDG-1 receptor (epithelial differentiation gene receptor). A sequence alignment of B1C3 with known receptors reveals that it is about 34% identical to mouse EDG-1 and 33% identical to rat EDG-1 receptors (FIG. 3).

### Example 2

### In Situ Hybridization Experiments

Preparation of tissue: Adult male Sprague-Dawley rats (about 250 gm; Charles River, St.-Constant, Quebec) were sacrificed by decapitation. Brain, spinal cord with DRGs still attached and several peripheral tissues (heart, kidney, spleen, liver, lung and skeletal muscle) were promptly removed, snap-frozen in isopentane at −40° C. for 20 sec. and stored at −80° C. Frozen tissue was sectioned at 16 _m in a Microm HM 500 MJ cryostat (Germany) and thaw-mounted onto ProbeOn PlusJ slides (Fisher Scientific, Montreal, Quebec). Sections were stored at −80° C. prior to in situ hybridization.

Riboprobe synthesis: The pBluescript KS⁻-B1A7 plasmid, containing a 1 Kb fragment of the 5′ end of the B1A7 gene, was linearized by cutting in the polylinker site on either side of the inserted cDNA using either NotI or SacI restriction enzymes (Pharmacia Biotech, U.S.A) and the appropriate buffers. Antisense and sense B1A7 riboprobes were transcribed in vitro using either T7 or T3 RNA polymerase (Promega), respectively, in the presence of _-[³⁵S]-UTP (about 800 Ci/mmol; Amersham, Oakville, Ontario

In situ Hybridization: Sections were postfixed in 4% paraformaldehyde (BDH, Poole, England) in 0.1 M phosphate buffer (pH 7.4) for 10 min at room temperature (RT) and rinsed in 3 changes of 2× standard sodium citrate buffer (SSC; 0.15 M NaCl. 0.015 M sodium citrate, pH 7.0). Sections were then equilibrated in 0.1 M triethanolamine, treated with 0.25% acetic anhydride in triethanolamine, rinsed in 2×SSC and dehydrated in an ethanol series (50-100%). Hybridization was performed in a buffer containing 75% formamide (Sigma, St-Louis, Mo.), 600 mM NaCl, 10 mM Tris (pH 7.5), 1 mM EDTA, 1× Denhardt's solution (Sigma), 50 _g/ml denatured salmon sperm DNA (Sigma), 10% dextran sulfate (Sigma), 20 mM dithiothreitol and _-[³⁵S]-UTP-labeled cRNA probes (10×10⁶ cpm/ml) at 55° C. for 18 h in humidified chambers. Following hybridization, slides were rinsed in 2×SSC at room temperature, treated with 20 _g/ml RNase IA (Pharmacia) in RNase buffer (10 mM Tris, 500 mM NaCl, 1 mM EDTA, pH 7.5) for 45 min at 37° C. and washed to a final stringency of 0.1×SSC at 70° C. Sections were then dehydrated with ethanol and exposed to Kodak Biomax MRJ film for 14-21 days and dipped in Kodak NTB2 emulsion diluted 1:1 with distilled water and exposed for 7-8 weeks at 4° C. prior to development and counterstaining with cresyl violet acetate (Sigma).

Results: In situ hybridization film autoradiograms reveal that B1A7 mRNA is expressed ubiquitously in the adult rat central nervous system (CNS). In the rat spinal cord, the B1A7 mRNA is highly expressed uniformly throughout the white and gray matter. In the rat brain, however, B1A7 mRNA is variably expressed. The receptor's mRNA is primarily expressed in fiber tracts throughout the brain, namely, the corpus callosum, the anterior commissure (anterior and posterior), fornix, fimbria (of the hippocampus), the internal capsule and the cerebellar peduncle. Other structures including the piriform cortex of the olfactory lobe, the hippocampus and the habenular nucleus are moderately labeled; while various other regions of the brain, such as the cortex and the periaqueductal gray, for example, mildly express message. Microscopic examination of emulsion processed sections suggests that these cells are non-neuronal.

In addition to this central distribution, a moderate hybridization signal for B1A7 message is detected in adult rat spleen, but not in heart, kidney, liver, lung or skeletal muscle. Preliminary results, using the rat B1A7 riboprobe, indicate that the B1A7 receptor mRNA is similarly distributed in the mouse CNS, while no labeling is detected in human adult spinal cords or DRGs.

### Example 3

### Development of an Assay for the Endogenous Ligand

One method that can be used to identify the endogenous ligand or to screen natural or synthetic compounds is to structurally link the receptor preferably through its carboxy terminus to a fluorescent probe, for example the green fluorescent protein (GFP). It is a well known fact that upon stimulation with an agonist, the GPCRs internalize (Ashworth et al., Proc. Nat. Acad. Sci. USA, 92:512-516 (1995)). When coupled to a fluorescent probe (e.g., GFP) one can easily monitor the internalization of the receptor by conventional techniques (e.g., fluorescent or confocal microscopy, etc.). Therefore, one can make the tissue extract from brain, spinal cord etc., incubate a fractionated extract with the cells expressing the hybrid GPCR-GFP construct and monitor internalization. Only the fractions containing endogenous ligand for this particular GPCR will cause the hybrid to internalize. Such an active fraction can then be further fractionated until the recovery of a pure ligand has been achieved. Similarly, one can assay for the receptor activating compounds in a complex mixture of synthetic library of compounds.

All references cited herein are fully incorporated herein by reference. Having now fully described the invention, it will be understood by those of skill in the art that the invention may be performed within a wide and equivalent range of conditions, parameters and the like, without affecting the spirit or scope of the invention or any embodiment thereof.

1. A protein, except as existing in nature, comprising the amino acid sequence consisting functionally of SEQ ID NO:1.

2. The protein of claim 1, wherein said amino acid sequence consists essentially of the amino acid sequence of SEQ ID NO:1.

3. An antibody made by a process comprising the step of injecting a pharmaceutically acceptable preparation comprising the protein of either claim 1 or claim 2 into an animal capable of producing said antibody.

4. The process of claim 3, wherein said animal is a mouse and said process further comprises fusing spleen cells from said mouse with myeloma cells to produce a monoclonal antibody binding to said protein.

5. An antibody that binds preferentially to the protein of claim 2.

6. A polynucleotide, except as existing in nature, encoding a protein comprising the amino acid sequence consisting functionally of the sequence of SEQ ID NO:1.

7. The polynucleotide of claim 6, wherein said polynucleotide encodes a protein consisting essentially of the amino acid sequence of SEQ ID NO:1.

8. An expression vector comprising the polynucleotide of either claim 6 or claim 7.

9. A host cell transformed with the vector of claim 8.

10. Recombinant B1C3 receptor produced by the host cell of claim 9.

11. The polynucleotide of claim 7, wherein said polynucleotide has a sequence consisting essentially of the nucleotide sequence of SEQ ID NO:2.

12. An expression vector comprising the polynucleotide of claim 11.

13. A host cell transformed with the vector of claim 12.

14. A method of assaying a test compound for its ability to bind to the B1C3 receptor, comprising:
a) incubating a source containing said B1C3 receptor with:
i) a ligand known to bind to said B1C3 receptor;
ii) said test compound; and
b) determining the extent to which said ligand binding is displaced by said test compound.

15. The method of claim 14, wherein said B1C3 receptor has the sequence shown in SEQ ID NO:1.

16. A method for determining if a test compound is an agonist of a B1C3 receptor, comprising:
a) incubating a cell expressing said B1C3 receptor with said test compound; and
b) determining whether said test compound causes a statistically significant increase in either intracellular adenyl cyclase activity or the intracellular concentration of calcium.

17. The method of claim 16, wherein said B1C3 receptor has the sequence shown in SEQ ID NO:1.

18. A method for determining if a test compound is a ligand or agonist of a B1C3 receptor, comprising determining whether a cell expressing said receptor internalizes it in response to contact with said test compound.

19. The method of claim 18, wherein:
a) said B1C3 receptor is recombinantly expressed in said cell as a fusion protein linked to a fluorescent protein;
b) the cell of step (a) is contacted with said test compound; and
c) internalization of receptor is determined by microscopy.

20. A method for determining if a test compound is an antagonist of an B1C3 receptor, comprising:
a) incorporating a DNA molecule encoding said B1C3 receptor into an expression vector so that it is operably linked to a promoter;
b) transfecting said expression vector into a host;
c) selecting cells transfected in step b) that have constitutively activated B1C3 receptors as evidenced by either:
i) a statistically significant increase in intracellular adenyl cyclase activity; or
ii) a statistically significant increase in intracellular calcium concentration;
d) contacting the cells selected in step c) with said test compound; and
e) determining if said test compound causes a statistically significant decrease in either said adenyl cyclase activity or said calcium concentration relative to control cells not contacted with said test compound.

21. The method of claim 20, wherein said B1C3 receptor has the sequence shown in SEQ ID NO:1.

22. A method for assaying a test compound for its ability to alter the activity of an B1C3 receptor, comprising:
a) incubating a source containing said B1C3 receptor with:
i) a ligand that binds with specificity to said B1C3 receptor;
ii) said test compound; and
b) determining whether said test compound increases or decreases intracellular calcium concentration in response to said ligand.

23. The method of claim 22, wherein said B1C3 receptor has the sequence shown in SEQ ID NO:1.

24. A method for assaying a test compound for its ability to alter the expression of a B1C3 receptor, comprising:
a) growing cells expressing said MP-10 receptor;
b) collecting said cells; and
c) comparing receptor expression in the cells exposed to said test compound with control cells grown under essentially identical conditions but not exposed to said test compound.

25. The method of claim 24, wherein said cells expressing said B1C3 receptor are cells transformed with an expression vector comprising a polynucleotide sequence encoding a protein with an amino acid sequence consisting essentially of the sequence shown in SEQ ID NO:1.

26. The method of any one of claims 24 or 25, wherein said test compound is an oligonucleotide at least 15 nucleotides in length and comprising a sequence complementary to the sequence of said B1C3 receptor.
